(19) United States
(12) Patent Application Publication
(10) Pub. No.: US 2025/0185934 A1
(43) Pub. Date: Jun. 12, 2025

(54) BIOLOGICAL INFORMATION MEASUREMENT DEVICE

(71) Applicant: OMRON HEALTHCARE Co., Ltd., Kyoto (JP)

(72) Inventor: Yukiya SAWANOI, Kyoto (JP)

(21) Appl. No.: 19/058,902

(22) Filed: Feb. 20, 2025

Related U.S. Application Data
(63) Continuation of application No. PCT/JP2023/028225, filed on Aug. 2, 2023.

(30) Foreign Application Priority Data
Dec. 1, 2022 (JP) 2022-193153

(51) Int. Cl. A61B 5/024; A61B 5/00

(52) U.S. Cl. CPC A61B 5/02444; A61B 5/0053; A61B 5/7271; A61B 5/742; A61B 2560/0462; A61B 2562/0247

(57) ABSTRACT

A biological information measurement device includes: pulse acquisition means configured to detect a pulse of a user; pulse interval calculation means configured to calculate a pulse interval between one beat and a beat immediately before the one beat based on the pulse; display means configured to display a level indicator visually indicating the pulse interval; and a display resolution determination unit configured to determine a minimum value and a maximum value of the pulse interval indicated by the level indicator using information on the pulse interval.

### CROSS-REFERENCE TO RELATED APPLICATIONS

This application is the U.S. national stage application filed pursuant to U.S.C. 365 (c) and 120 as a continuation of International Patent Application No. PCT/JP2023/028225, filed Aug. 2, 2023, which application claims priority to Japanese Patent Application No. 2022-193153, filed Dec. 1, 2022, which applications are incorporated herein by reference in their entireties.

### TECHNICAL FIELD

The present invention relates to a biological information measurement device for measuring pulses of a living body, and more particularly to a biological information measurement device for providing information related to an interval of measured pulses.

### BACKGROUND ART

In recent years, it has become widespread to perform health management by measuring information related to the body and health of an individual, such as a blood pressure value, with a measurement device and recording and analyzing the measurement result. In particular, arrhythmia such as atrial fibrillation (AF) may lead to cerebral and cardiovascular diseases. Thus, it is effective to detect a fluctuation in a pulse interval with the above-described device and notify a user so that the user can easily recognize the fluctuation.

In the related art, it has been known to provide information related to such a pulse interval based on biological information acquired when blood pressure measurement is performed using a blood pressure monitor. For example, Patent Literature 1 discloses a blood pressure monitor capable of storing a pulse wave used for measurement of a blood pressure value and displaying a pulse wave graph simultaneously with the blood pressure value. Patent Literature 1 also discloses that a heart mark displayed on a screen blinks in accordance with pulsation during blood pressure value calculation.

According to the blood pressure monitor described in Patent Literature 1, a user can recognize a pulse interval by checking a blinking interval of the heart mark blinking in accordance with pulsation during blood pressure measurement. Since a time-series graph of signal levels of pulse waves is subsequently displayed as a time-series pulse wave graph, it is possible to check a pulse interval (and a fluctuation thereof) by reading such a graph.

### CITATION LIST

### Patent Literature

Patent Literature 1: JP 2007-98003 A

### SUMMARY OF INVENTION

### Technical Problem

However, only blinking a mark in accordance with pulsation as in the technique described in Patent Literature 1 is not enough to easily recognize a fluctuation in a pulse interval, and there is a problem in that an important fluctuation in a pulse interval is overlooked. In this respect, for example, it is conceivable to display a pulse interval for each beat with a level indicator such as a so-called bar graph meter instead of blinking of the mark, so that the fluctuation in the pulse interval can be easily recognized.

A pulse interval varies greatly among individuals, and the magnitude of the interval and the average value thereof vary among individuals. For example, in a general blood pressure monitor, the range of a pulse rate to be measured is 40 beats/minute to 180 beats/minute, and an average pulse interval is 1.5 seconds for a user with a pulse rate of 40 beats/minute and 0.3 seconds for a user with a pulse rate of 180 beats/minute.

For this reason, in order to display a fluctuation in a pulse interval during measurement in real time by the level indicator as described above, it is necessary to set the maximum value of the level indicator such that an assumed maximum pulse interval (e.g., 1.5 seconds) can be displayed. As a result, there is a problem in that the display resolution of the level indicator becomes low for a user with a small pulse interval (i.e., a high pulse rate), and it becomes difficult for the user to recognize a fluctuation in a pulse interval.

In view of the above-described circumstances, an object of the present invention is to provide a technique for optimizing the resolution of a display region indicating a pulse interval in a measurement device capable of detecting pulses, and making it easy to visually recognize a fluctuation in a pulse interval regardless of an individual difference in the pulse interval.

### Solution to Problem

The present invention employs the following configurations to solve the above-described problems. That is, a biological information measurement device includes:
pulse acquisition means configured to detect a pulse of a user;
pulse interval calculation means configured to calculate a pulse interval between one beat and a beat immediately before the one beat based on the pulse;
display means configured to display a level indicator visually indicating the pulse interval; and
a display resolution determination unit configured to determine a minimum value and a maximum value of the pulse interval indicated by the level indicator using information on the pulse interval.

The level indicator described here is only required to indicate a predetermined feature amount by using a non-numerical display (e.g., the size of a display region), and the shape and display mode thereof are not limited. For example, the level indicator may visually indicate the pulse interval or the amount of change by using at least any of a length, an area, an angle of a region in which display is activated on the display means, or the number of regions in which display is activated on the display means.

With such a configuration, the maximum value and the minimum value of the pulse interval displayed at the level indicator can be optimized for the user, so that a fluctuation in the pulse interval can be easily visually recognized regardless of an individual difference in the pulse interval.

The display resolution determination unit may determine a value obtained by multiplying a maximum value of a plurality of the pulse intervals calculated and being consecutive by a predetermined first ratio as the maximum value of the pulse interval indicated by the level indicator. With such a configuration, since a value obtained by providing a predetermined margin from an actually measured maximum pulse interval of the user can be set as the maximum value at the level indicator, the resolution of the level indicator can be determined with reference to the maximum value optimized for the user.

The display resolution determination unit may determine a value obtained by multiplying a minimum value of a plurality of the pulse intervals calculated and being consecutive by a predetermined second ratio as the minimum value of the pulse interval indicated by the level indicator. With such a configuration, since a value obtained by providing a predetermined margin from an actually measured minimum pulse interval of the user can be set as the minimum value in the level indicator, the resolution of the level indicator can be optimized for the user with reference to the minimum value and the maximum value optimized for the user.

The display resolution determination unit may calculate an average value of a plurality of the pulse intervals calculated and being consecutive, determine a value obtained by multiplying a positive maximum deviation from the average value by a third ratio as the maximum value of the pulse interval indicated by the level indicator, and determine a value obtained by multiplying a negative maximum deviation from the average value by a fourth ratio as the minimum value of the pulse interval indicated by the level indicator.

With such a configuration, the resolution of the level indicator can be optimized for the user by setting values obtained by providing predetermined margins to the positive and negative maximum deviations from the average value of the actually measured pulse intervals, as the minimum value and the maximum value of the level indicator. Further, by changing the third ratio and the fourth ratio such that the average value becomes the median value of the level indicator instead of setting the third ratio and the fourth ratio to fixed values, it is possible to realize display of the level indicator with improved visibility.

The pulse acquisition means may include a cuff and a pressure sensor and may detect the pulse by pressurizing a blood vessel of the user with the cuff and detecting a pressure pulse wave of the blood vessel with the pressure sensor, and the display resolution determination unit may determine the minimum value and the maximum value of the pulse interval indicated by the level indicator using information on a plurality of the pulse intervals calculated from a start of pressurization of the blood vessel until a first predetermined condition is satisfied.

Here, the first predetermined condition may be, for example, that a predetermined time (e.g., 5 seconds) has elapsed from the start of pressurization of the blood vessel with the cuff, or that a cuff pressure reaches a predetermined pressure (e.g., 40 mHg). With such a configuration, information on the plurality of consecutive pulse intervals for determining the maximum value and the minimum value of the level indicator can be acquired without performing a separate measurement both in pressurization measurement and in depressurization measurement.

The biological information measurement device may further include storage means configured to store information on the pulse interval detected, in which the pulse acquisition means may include a cuff and a pressure sensor and may detect the pulse by pressurizing a blood vessel of the user with the cuff and detecting a pressure pulse wave of the blood vessel with the pressure sensor, and the display resolution determination unit may determine the minimum value and the maximum value of the pulse interval indicated by the level indicator using the information on the pulse interval stored in the storage means and satisfying a second predetermined condition.

Here, the second predetermined condition may be, for example, all pulse intervals calculated at the time of the most recent biological information measurement. With such a configuration, the minimum value and the maximum value of the pulse interval indicated by the level indicator can be determined with high accuracy by using a sufficient amount of information on the pulse interval of the user.

The pulse acquisition means may include a cuff and a pressure sensor and may detect the pulse by pressurizing a blood vessel of the user with the cuff and detecting a pressure pulse wave of the blood vessel with the pressure sensor, and the display resolution determination unit may determine the minimum value and the maximum value of the pulse interval indicated by the level indicator using information on a plurality of the pulse intervals being consecutive and calculated until a fluid supplied to the cuff is discharged.

With such a configuration, in the depressurization measurement, the minimum value and the maximum value of the pulse interval indicated by the level indicator can be determined with high accuracy by using a sufficient amount of information on the pulse interval of the user acquired in a pressurization step.

The level indicator may be made up of a plurality of display segments, and may represent the pulse interval by the number of the display segments in which display is activated.

Here, “activation of display” refers to a transition to a display state in a region that can be switched between a display state and a non-display state (that is, deactivation of display refers to a transition to a non-display state). For example, when the display means is an LCD, “activation of display” corresponds to outputting display in a display region on the display of the LCD, and when the display means is an LED light or the like, “activation of display” corresponds to the light being turned on. Each display segment is a unit of a display region that can be individually switched between a display activation state and a display deactivation state, and a shape thereof is not particularly limited.

The configurations and processing described above can be combined with one another to constitute the present invention as long as technical contradiction does not occur. Further, in the above description, the first to fourth ratios may be different values or may be the same value.

### Advantageous Effects of Invention

According to the present invention, it is possible to provide a technique for optimizing the resolution of a display region indicating a pulse interval in a measurement device capable of detecting pulses, and making it easy to visually recognize a fluctuation in a pulse interval regardless of an individual difference in the pulse interval.

### BRIEF DESCRIPTION OF DRAWINGS

FIG. 1 is a schematic diagram illustrating an outline of a device configuration and a functional configuration of a blood pressure measurement device according to Example 1.

FIG. 2(A) is a first diagram illustrating an example of a level indicator displayed on image display means of the blood pressure measurement device according to Example 1. FIG. 2(B) is a second diagram illustrating an example of the level indicator displayed on the image display means of the blood pressure measurement device according to Example 1. FIG. 2(C) is a third diagram illustrating an example of the level indicator displayed on the image display means of the blood pressure measurement device according to Example 1.

FIG. 3 is an explanatory diagram for describing a pulse wave signal and a pulse interval detected during blood pressure measurement.

FIG. 4 is a graph illustrating an example of variation in a pulse interval between a person with sinus rhythm and a person with atrial fibrillation.

FIG. 5 is an explanatory diagram for describing the display resolution of the level indicator of the blood pressure measurement device according to Example.

FIG. 6 is a flowchart illustrating an example of processing performed in the blood pressure measurement device according to Example.

FIG. 7 is a flowchart illustrating a flow of processing performed in a third modified example of Example 1.

FIG. 8 is a flowchart illustrating a flow of processing performed in a fourth modified example of Example 1.

FIG. 9 is a flowchart illustrating a flow of processing performed in a fifth modified example of Example 1.

FIG. 10 is a flowchart illustrating a flow of processing performed in a sixth modified example of Example 1.

FIG. 11(A) is a first diagram illustrating a variant of the level indicator displayed on the image display means of the blood pressure measurement device according to Example. FIG. 11(B) is a second diagram illustrating a variant of the level indicator displayed on the image display means of the blood pressure measurement device according to Example. FIG. 11(C) is a third diagram illustrating a variant of the level indicator displayed on the image display means of the blood pressure measurement device according to Example.

FIG. 12(A) is a fourth diagram illustrating a variant of the level indicator displayed on the image display means of the blood pressure measurement device according to Example. FIG. 12(B) is a fifth diagram illustrating a variant of the level indicator displayed on the image display means of the blood pressure measurement device according to Example. FIG. 12(C) is a sixth diagram illustrating a variant of the level indicator displayed on the image display means of the blood pressure measurement device according to Example.

FIG. 13(A) is a seventh diagram illustrating a variant of the level indicator displayed on the image display means of the blood pressure measurement device according to Example. FIG. 13(B) is an eighth diagram illustrating a variant of the level indicator displayed on the image display means of the blood pressure measurement device according to Example.

### DESCRIPTION OF EMBODIMENTS

### Example 1

Examples of the present invention will be described below with reference to the drawings. Note that the material, shape, relative arrangement, and the like of configurations described in these examples are not intended to limit the scope of the present invention to the configurations alone, unless otherwise stated.

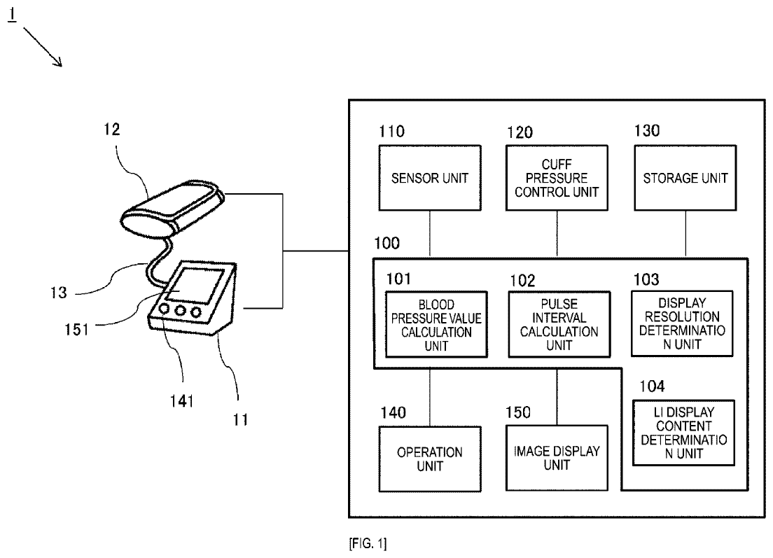

The present invention can be applied to, for example, a blood pressure measurement device 1 as illustrated in FIG. 1. FIG. 1 is a schematic diagram illustrating an outline of a device configuration and a functional configuration of the blood pressure measurement device 1 according to the present example. As illustrated in FIG. 1, the blood pressure measurement device 1 generally includes a main body portion 11, a cuff portion 12, and an air tube 13. As indicated by the functional blocks in FIG. 1, the blood pressure measurement device 1 includes functional units of a control unit 100, a sensor unit 110, a cuff pressure control unit 120, a storage unit 130, an operation unit 140, and an image display unit 150.

The main body portion 11 includes image display means 151 such as a liquid crystal display (LCD), various operation buttons 141, as well as, although not illustrated, sound output means such as a speaker, a power supply unit such as a battery, a pump and a valve communicating with the cuff portion, a housing accommodating these components, and the like. The cuff portion 12 is a member used by being wrapped around an upper arm of a user, and includes an air bag (cuff) communicating with the pump and the valve of the main body portion 11 via the air tube 13, a belt incorporating the cuff, a pressure sensor provided at the belt (none of which are illustrated), and the like. When blood pressure measurement is performed by the Korotkoff method, a microphone may be included.

The belt of the cuff portion 12 is provided with fixing means (e.g., a hook-and-loop fastener) for fixing the cuff portion 12 to the upper arm of the user, and the cuff portion 12 is wrapped around the upper arm of the user by the belt when blood pressure measurement is performed using the blood pressure measurement device 1.

The control unit 100 is means for controlling the blood pressure measurement device 1, and includes, for example, a central processing unit (CPU). Upon receiving operation by the user via the operation unit 140, the control unit 100 controls each component of the blood pressure measurement device 1 to execute various types of processing such as blood pressure measurement and provision of various types of information in accordance with a predetermined program. The predetermined program is stored in and read from the storage unit 130 to be described below. The control unit 100 includes a blood pressure value calculation unit 101, a pulse interval calculation unit 102, a display resolution determination unit 103, and a level indicator (LI) display content determination unit 104 as functional modules. These functional modules will be described in detail below.

The sensor unit 110 includes the pressure sensor (e.g., a piezoresistive sensor including a piezoelectric element) provided at the cuff portion 12 as described above, and detects at least a pulse wave of the user. The sensor unit 110 may include a sensor other than the pressure sensor, and may include a photoplethysmography (PPG) sensor when a pulse wave is detected by a photoelectric method. The blood pressure measurement device 1 according to the present example acquires a pulse of the user based on a pulse wave detected by the sensor unit 110. That is, in the present example, the cuff and the sensor unit 110 (including the pressure sensor) at the cuff portion 12 correspond to the pulse acquisition means.

The cuff pressure control unit 120 controls the pump and the valve of the main body portion 11 to adjust a cuff pressure of the cuff portion 12 at the time of blood pressure measurement. Specifically, at the time of blood pressure measurement, control is performed such that the pump is driven in a state where the cuff portion 12 is wrapped around the upper arm to feed air into the cuff so as to inflate the cuff (increase the cuff pressure). In this way, the blood flow is once blocked by compressing the blood vessel of the upper arm of the user, and then control is performed such that the pump is stopped and the valve is opened to gradually discharge air from the cuff so as to deflate the cuff (decrease the cuff pressure). Hereinafter, a step of increasing the cuff pressure is also referred to as a cuff inflation step, and a step of decreasing the cuff pressure is also referred to as a cuff deflation step.

The storage unit 130 includes a main storage device such as a random access memory (RAM), a read only memory (ROM), or the like, and an auxiliary storage device such as a HDD, a flash memory, or the like, and stores various types of information such as application programs, various type of measurement results such as a blood pressure value, a pulse rate, a pulse wave, and a pulse interval for each beat, and other biological information acquired. A measured blood pressure value, a measured pulse rate, and the like is stored in the storage unit 130 in association with time information such as an acquisition time or a measurement time. As the time information, for example, information measured with reference to a real time clock (RTC) can be used. The auxiliary storage device may be configured to be attachable to and detachable from the main body portion 11.

The operation unit 140 includes various operation buttons 141 (e.g., a power button, a measurement execution button, and a selection and determination button), and has a function of receiving input operation from the user and causing the control unit 100 to execute processing in accordance with the operation.

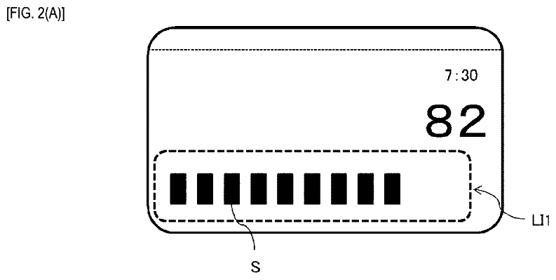

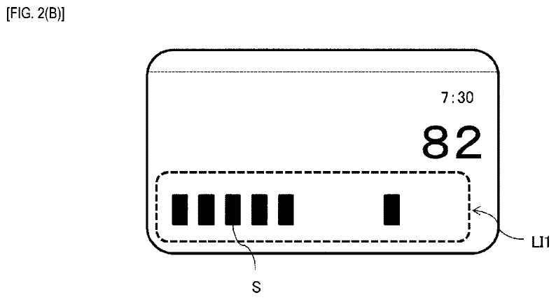

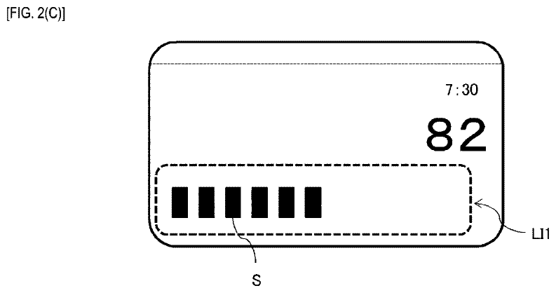

The image display unit 150 includes the image display means 151 of the main body portion 11, and provides the user with information by displaying various types of information such as measured blood pressure values, the current time, and information regarding a cuff attachment state on the image display means 151. FIG. 2(A) to FIG. 2(C) illustrate examples of the display content of the image display means 151. As illustrated in FIG. 2(A) to FIG. 2(C), the image display means 151 is provided with a region for displaying a level indicator LI1 indicating information about a pulse interval to be described below. The level indicator LI1 will be described in further detail below.

Hereinafter, each functional module of the control unit 100 will be described. The blood pressure value calculation unit 101 calculates a blood pressure value (and a pulse rate) of the user based on a pulse wave acquired by the sensor unit 110. As a blood pressure calculation method, any desired known technique can be used and, for example, an oscillometric method in which a blood pressure is measured by detecting a pressure pulse wave with a pressure sensor can be employed. A microphone may be provided at the cuff portion 12 and the Korotkoff method for detecting Korotkoff sounds may be used. The blood pressure value and the pulse rate calculated by the blood pressure value calculation unit 101 may be stored in the storage unit 130 in association with a blood pressure measurement time.

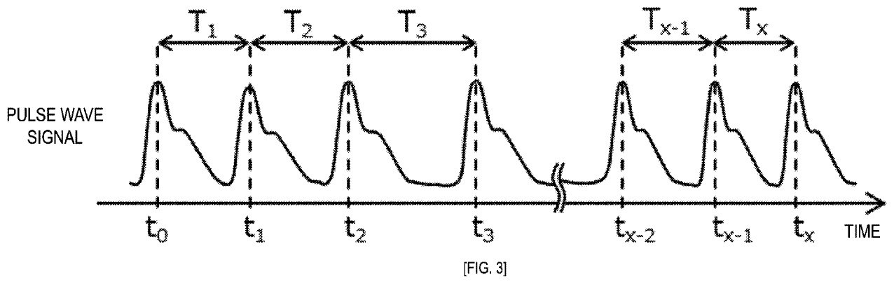

The pulse interval calculation unit 102 calculates, from a waveform of the pulse wave acquired by the sensor unit 110 (e.g., a pressure pulse wave acquired by the pressure sensor), an inter-peak time interval of the pulse wave for each beat. The calculation of the pulse interval will be described with reference to FIG. 3. FIG. 3 is an explanatory diagram schematically illustrating a relationship between a pulse wave signal and a time. In FIG. 3, when the time at which the peak of a certain wave is detected is defined as to and the time at which the peak of the next wave is detected is defined as t₁, the interval between the certain wave and the next wave is t₁-t₀=T1. In this way, the pulse interval calculation unit 102 calculates tₓ-tₓ₋₁=Tₓ as the pulse interval.

The display resolution determination unit 103 determines the minimum value and the maximum value of the pulse interval displayed at the level indicator LI1 using information on the pulse interval calculated by the pulse interval calculation unit 102. Here, the display content of the level indicator LI1 will be described with reference to FIG. 2(A) to FIG. 2(C). The level indicator LI1 according to the present example includes a plurality of display segments S capable of being switched between a display state and a non-display state, and can indicate the magnitude of the pulse interval calculated by the pulse interval calculation unit 102 by the number of the display segments S in a state in which the display is activated (in the display state, not in the non-display state).

The level indicator LI1 in the present example is provided with ten display segments S, and the level indicator display content determination unit 104, which will be described below, determines how many display segments among the ten display segments S are to be activated, and the determined display segments S are displayed at the level indicator LI1, whereby the magnitude of the pulse interval can be visually indicated.

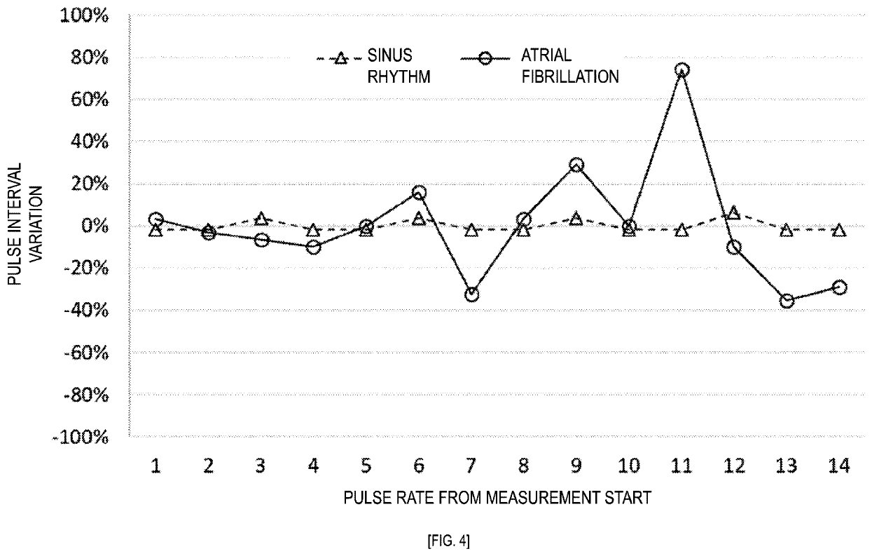

A pulse interval varies greatly among individuals, and the magnitude of the interval and the average value thereof vary among individuals. In addition, as illustrated in FIG. 4, variation in a pulse interval is larger in a person with atrial fibrillation than in a person with sinus rhythm (healthy person). FIG. 4 is a graph illustrating an example of variation in a pulse interval between a person with sinus rhythm person and person with atrial fibrillation, in which the horizontal axis (X-axis) represents the pulse rate from the start of measurement and the vertical axis (Y-axis) represents variation in a pulse interval. The variation in the pulse interval is indicated by a ratio (%) to an average pulse interval. In the graph illustrated in FIG. 4, the variation in the pulse interval is 10% or less in the person with sinus rhythm, while reaching about 80% in the person with atrial fibrillation.

For this reason, when the upper and lower limit values of the pulse interval displayed by the level indicator LI1 are set as predetermined fixed values, the range between the upper and lower limit values has to be large, and thus the display resolution of the level indicator LI1 becomes insufficient for a user with a high pulse rate (i.e., a user with a short pulse interval). For example, when ±100% of the average pulse interval are set as the upper and lower limit values of the pulse interval displayed by the level indicator LI1 in consideration of the variation in the pulse interval of the above-described person with atrial fibrillation, and the average pulse interval is estimated to be 1.5 seconds (i.e., large) based on a minimum pulse rate of 40 beats/m that can be measured by a general blood pressure monitor, the pulse interval indicated by one display segment S is 0.3 seconds. That is, the upper and lower limit values of the pulse interval displayed at the level indicator LI1 are (0.3 seconds to 3 seconds).

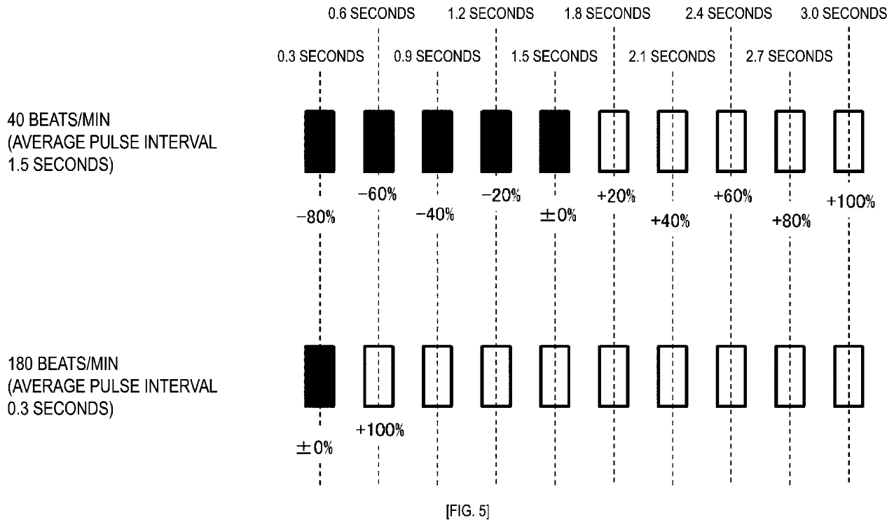

However, with the upper and lower limit values assuming a user with a low pulse rate as described above, the pulse interval of a user with a high pulse rate (e.g., 180 beats/m, where the average pulse interval is 0.3 seconds) cannot be appropriately represented. FIG. 5 illustrates a comparison relationship between a display indicating an average pulse interval of a user with a pulse rate of 40 beats/m and a display indicating an average pulse interval of a user with a pulse rate of 180 beats/m on the assumption that the number of the display segments is 10 and the upper and lower limit values of the pulse interval displayed at the level indicator LI1 are set to 0.3 to 3 seconds.

In this regard, the display resolution determination unit 103 according to the present example determines the minimum value and the maximum value of the pulse interval displayed at the level indicator LI1 by using information on an actually measured pulse interval for each user. Accordingly, the display resolution of the level indicator LI1 can be dynamically set, and a fluctuation in the pulse interval can be expressed with good visibility regardless of the magnitude of the pulse rate of the user. A specific method for determining the minimum value and the maximum value of the pulse interval will be described below.

The level indicator display content determination unit 104 determines the display content of the level indicator LI1 displayed on the image display means 151. Specifically, the level indicator display content determination unit 104 determines the number of the display segments S to be activated based on the pulse interval Tₓ calculated for each beat, and determines the display content at the level indicator LI1 for each beat.

Here, a specific example in which the level indicator display content determination unit 104 determines the number of the display segments S to be activated will be described. The level indicator display content determination unit 104 obtains the number Nₓ of display segments S corresponding to the calculated pulse interval by Equations (1) and Equation (2) described below, where Tₘᵢₙ, Tₘₐₓ, and Nₘₐₓ are the minimum value of the pulse interval, the maximum value of the pulse interval, and the maximum number of display segments S which are determined by the display resolution determination unit 103, respectively.

$$\begin{matrix}[ Equation 1 ] & \\ Δ t = ( {T}_{max} - {T}_{min} ) / ( {N}_{max} - 1 ) & ( 1 )\end{matrix}$$

$$\begin{matrix}[ Equation 2 ] & \\ {N}_{x} = ⌊ ( {T}_{x} - {T}_{min} ) / Δ t + 1 ⌋ & ( 2 )\end{matrix}$$

That is, Nₓ is the maximum integer not exceeding the value of (Tₓ-Tₘᵢₙ)/Δt+1. By obtaining Nₓ in this way and displaying Nₓ display segments S at the level indicator LI1 for each beat, it is possible to indicate a fluctuation in the pulse interval. Specifically, when the number of the display segments S in which the display is activated is large, a display-activated region of the level indicator LI1 becomes large, and conversely, when the number of the display segments S in which the display is activated is small, the display-activated region of the level indicator LI1 becomes small. That is, the display-activated region of the level indicator LI1 becomes larger as the pulse interval becomes larger (longer), and the display-activated region of the level indicator LI1 becomes smaller as the pulse interval becomes smaller (shorter). Thus, the user can intuitively recognize a variation in the size of the display-activated region by viewing the display, thereby recognizing an amount of change in the pulse interval for each beat.

Contriving a display mode enables the display segments S displayed at the level indicator LI1 to indicate the amount of change in the pulse interval to the user in an easy-to-understand manner. For example, in a mode in which the display segments S are arranged in a straight line extending in the left-right direction as illustrated in FIG. 2(A) to FIG. 2(C), the display activation of the display segments S starts from the leftmost display segment S, the number of the display segments S to be displayed is sequentially increased rightward one by one until Nₓ display segments S are displayed, and after the Nₓ display segments S are displayed, the display segments S are sequentially hidden from the right.

FIG. 2(A) to FIG. 2(C) illustrate an example of such a display transition of the display segments S. FIG. 2(A) illustrates the level indicator LI1 in a case where Nₓ is 9 as an indication of the pulse interval Tₓ at a certain point of time. In this example, Nₘₐₓ is 10. That is, a state is illustrated in which there are 10 display segments S in the level indicator LI1 as a whole, and 9 display segments S are displayed (activated) in a left-aligned manner.

FIG. 2(B) illustrates a state in which the display segments S are sequentially hidden from the right. As illustrated in FIG. 2(B), the display of the Nₓ-th display segment S (in this case, the ninth display segment S) remains activated when the display segments S are sequentially hidden, whereby it is possible to make it easy to recognize a fluctuation in the pulse interval from a change in the position of the remaining display segment S. However, this is not necessarily required, and the display of all the Nₓ display segments S may be simultaneously activated and all the Nₓ display segments S may remain displayed until the next pulse interval is obtained.

When the peak of the next pulse wave is detected and the level indicator display content determination unit 104 determines the display content of the pulse interval Tₓ+1 calculated based on the peak of the next pulse wave, six display segments S are displayed in an activated state at the level indicator LI1 as illustrated in FIG. 2(C). In the present example, the display of the level indicator LI1 changes in synchronization with the waveform of the detected pulse. That is, when the pulse interval is short, the timing at which the display of the level indicator LI1 changes is advanced accordingly, and when the pulse interval is long, the timing at which the display changes is delayed. However, the timing at which the display of the level indicator LI1 changes does not necessarily need to be synchronized with the pulse wave, and it is possible to appropriately set timings at which the display indicating the pulse interval starts and ends for each beat.

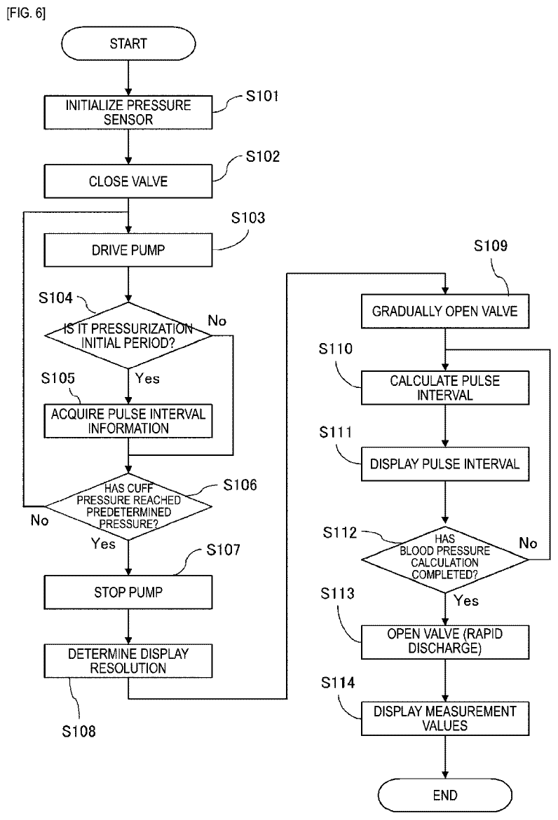

Next, with reference to FIG. 6, a flow of processing performed when blood pressure measurement is performed by the blood pressure measurement device 1 according to the present example and a fluctuation in the pulse interval of the user during the blood pressure measurement is displayed by the level indicator LI1 will be described. FIG. 6 is a flowchart illustrating a flow of processing performed when blood pressure measurement is performed by the blood pressure measurement device 1 according to the present example.

As illustrated in FIG. 6, when the user performs an operation to start blood pressure measurement, the sensor unit 110 initializes the pressure sensor (S101). Subsequently, the cuff pressure control unit 120 closes the valve (S102) and drives the pump (S103) to feed air into the cuff so as to inflate the cuff. Then, the control unit 100 determines whether the current time point is a predetermined pressurization initial period (S104). Whether the current time point is the predetermined pressurization initial period can be determined based on a condition such as whether a predetermined time (e.g., 5 seconds) has elapsed from the start of a measurement operation, or whether the cuff pressure has reached a predetermined value (e.g., 40 mHg), for example.

When it is determined in step S104 that the current time point is the predetermined pressurization initial period, information on the pulse interval calculated by the pulse interval calculation unit 102 is acquired (S105). Specifically, for example, information on the pulse interval calculated for each beat is accumulated in the storage unit 130. When the process of step S105 is executed, the processing proceeds to step S106, and it is determined whether the cuff pressure has reached a predetermined pressure (e.g., 200 mHg) (S106). On the other hand, when it is determined in step S104 that the current time point is not the predetermined pressurization initial period, the processing proceeds directly to S106.

When it is determined in step S106 that the cuff pressure has not reached the predetermined pressure, the processing returns to step S103, and the subsequent processing is repeated. On the other hand, when it is determined in step S106 that the cuff pressure has reached the predetermined pressure, the cuff pressure control unit 120 stops the pump (S107).

Then, the display resolution determination unit 103 determines the maximum value and the minimum value of the pulse interval displayed at the level indicator LI1 based on information on a plurality of consecutive pulse intervals acquired in S105. That is, the display resolution of the level indicator LI1 is determined (S108). Specifically, for example, a value obtained by multiplying the minimum value among the acquired plurality of consecutive pulse intervals by a preset ratio (e.g., 90%) can be determined to be the minimum value of the pulse interval indicated by the level indicator LI1. A value obtained by multiplying the maximum value among the acquired plurality of consecutive pulse intervals by a preset ratio (e.g., 110%) can be determined to be the maximum value of the pulse interval indicated by the level indicator LI1. Since the display resolution determined in this manner is determined based on the actually acquired pulse interval of the user, the display resolution is optimized for the user.

Following the process of step S108, the cuff pressure control unit 120 gradually opens the valve to deflate the cuff (S109). Then, the pulse interval calculation unit 102 calculates a pulse interval for each beat from a pulse wave acquired during this period (S110), and based thereon, the level indicator display content determination unit 104 determines the number of the display segments S to be activated for each beat. Then, the image display unit 150 displays the determined content at the level indicator LI1 (S111), so that the user can easily visually recognize a fluctuation in the pulse interval for each beat during the blood pressure measurement.

In addition, the blood pressure value calculation unit 101 measures blood pressure values (a systolic blood pressure and a diastolic blood pressure) in the course of deflation of the cuff, and in step S112, the blood pressure value calculation unit 101 determines whether the blood pressure value calculation has been completed (S112). Here, when it is determined that the blood pressure value calculation has not been completed, the processing returns to step S110, and the subsequent processing is repeated. On the other hand, when it is determined in step S112 that the blood pressure value calculation has been completed, the cuff pressure control unit 120 opens the valve to rapidly discharge air from the cuff to deflate the cuff (S113). Then, the image display unit 150 displays measurement values (which may include a pulse rate and the like in addition to the blood pressure values) (S114), and a series of processing ends. The various measured values may be stored in the storage unit 130.

According to the blood pressure measurement device 1 having the above-described configuration, the user can intuitively recognize a fluctuation in the pulse interval during blood pressure measurement and can more easily recognize the beat of the pulse. Thus, when there is an abnormality such as arrhythmia, a pulse abnormality can be easily recognized from a sense of discomfort, which can contribute to early detection of a circulatory system disease through daily blood pressure measurement. Further, since the display resolution determination unit 103 determines the display resolution of the level indicator LI1 that is optimized for the user, a fluctuation in the pulse interval can be indicated with an appropriate resolution regardless of a difference in a pulse interval between users.

### Modified Example 1

In Example 1 described above, an example in which the values obtained by multiplying the minimum value and the maximum value among the plurality of consecutive pulse intervals acquired in step S105 by the preset ratios are determined to be the minimum value and the maximum value of the pulse interval indicated by the level indicator LI1 has been described, but this is not necessarily essential. Such processing may be performed for only one of the minimum value and the maximum value, and a specified value may be used for the other. For example, the display resolution determination unit 103 may determine a specified value (e.g., 0 seconds) as the minimum value of the pulse interval indicated by the level indicator LI1.

### Modified Example 2

Alternatively, the display resolution determination unit 103 may calculate an average value of the plurality of consecutive pulse intervals acquired in step S105, and may determine values obtained by multiplying positive and negative maximum deviations from the average value among the acquired plurality of consecutive pulse intervals by a predetermined ratio (e.g., 110%) as the minimum value and the maximum value of the pulse interval indicated by the level indicator LI1, respectively. The predetermined ratio to be multiplied may be different between the positive maximum deviation and the negative maximum deviation. Further, by changing the ratio to be multiplied to the positive and negative maximum deviations such that the average value becomes the median value of the level indicator instead of setting the ratio to be multiplied to the positive and negative maximum deviations to fixed value, it is possible to realize display of the level indicator with improved visibility.

### Modified Example 3

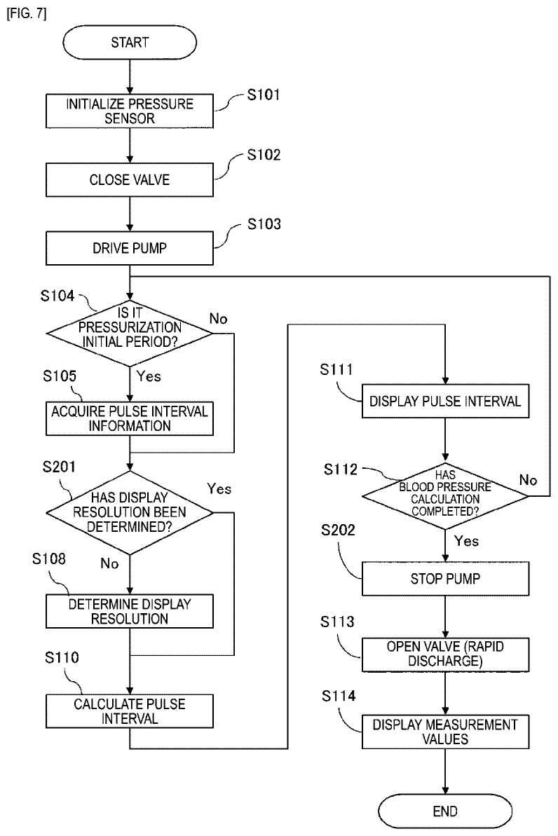

In Example 1 described above, the processing performed when blood pressure measurement is executed by a so-called depressurization measurement has been described, but the present invention can also be applied to a case where blood pressure measurement is executed by a so-called pressurization measurement method. The processing in such a case is illustrated in FIG. 7. Note that the device configuration of the biological information measurement device in the present modified example is the same as that in Example 1. In the processing illustrated in FIG. 7, the same steps as those described in Example 1 are denoted by the same reference signs, and a detailed description thereof are omitted.

As illustrated in FIG. 7, in the present modified example, the processing from step S101 to step S105 is the same as that in Example 1. In the present modified example, after step S105, the control unit 100 executes a process of determining whether the display resolution has been determined, that is, whether the minimum value and the maximum values of the pulse interval displayed by the level indicator LI1 have been optimized for the user (S201). Here, when it is determined that the display resolution has not been determined yet, the display resolution determination unit 103 executes a process of determining the display resolution (S108). The method for determining the display resolution may be the same as that in Example 1, or the method described in Modified Example 1 or 2 may be used.

Here, in the present modified example, since blood pressure measurement is performed by the pressurization measurement method, a process of calculating blood pressure values by the blood pressure value calculation unit 101 is executed together with the process of determining the display resolution. For this reason, when it is determined in step S201 that the display resolution has been determined, immediately after the display resolution is determined in step S108, the pulse interval calculation unit 102 calculates a pulse interval (S110) and the image display unit 150 displays the calculated pulse interval at the level indicator LI1 for each beat (S111). Then, the blood pressure value calculation unit 101 determines whether the blood pressure value calculation has been completed (S112), and when it is determined that the blood pressure value calculation has not been completed, the processing returns to step S104, and the subsequent processing is repeated. On the other hand, when it is determined in step S112 that the blood pressure value calculation has been completed, the cuff pressure control unit 120 stops the pump (S202) and opens the valve to deflate the cuff (S113). Then, the image display unit 150 displays the measurement values (S114), and a series of processing ends.

As described above, by determining the display resolution using information on the pulse interval obtained at the initial stage of the blood pressure measurement (more specifically, at the initial stage of pressurization by the cuff), not only in the depressurization measurement but also in the pressurization measurement, it is possible to optimize the display resolution of the level indicator LI1 for the user and to display a fluctuation in the pulse interval for each beat.

### Modified Example 4

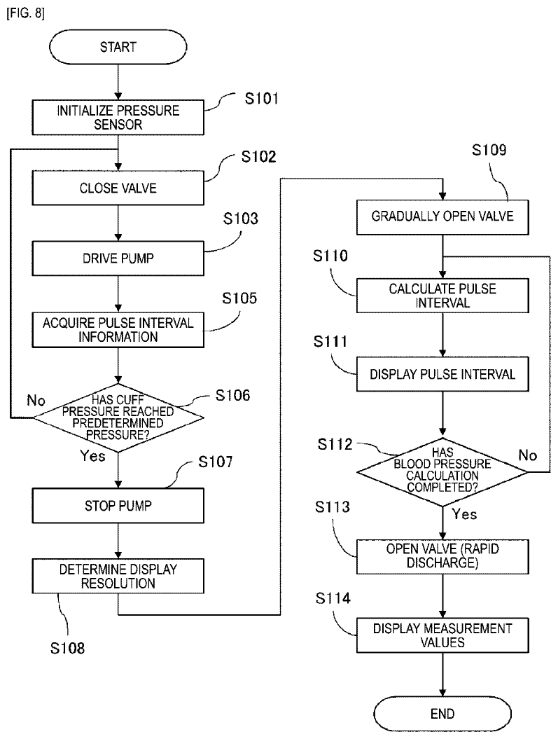

Only in a case where blood pressure measurement is performed by the depressurization measurement method, the processing flow as described below may be employed. FIG. 8 is a flowchart illustrating a part of a flow of processing executed in the blood pressure measurement device 1 in the present modified example. As illustrated in FIG. 8, the flow of the processing of the present modified example is the same as that of Example 1, except that the processing in step S104 in Example 1 is not present. That is, in the present modified example, the processing is performed such that pulse intervals are consecutively acquired during a cuff inflation stage for the depressurization measurement, a display resolution is determined using information on the acquired plurality of consecutive pulse intervals, and then a pulse interval calculated in a cuff deflation stage for the blood pressure measurement is displayed at the level indicator LI1 for each beat. With this configuration, since the amount of data that can be used for determining the display resolution is larger than when only the information on the pulse interval acquired at the initial stage of the cuff pressurization is used, the display resolution can be determined with higher accuracy.

### Modified Example 5

Next, a fifth modified example according to the present invention will be described. The device configuration of the blood pressure measurement device 1 according to the present modified example is the same as that of the blood pressure measurement device 1 of Example 1, and thus the same reference numerals as those in Example 1 are used, and redundant portions are omitted. In the present modified example, the processing related to display resolution determination is different from that of Example 1, and thus this point will be mainly described.

In the blood pressure measurement device 1 according to the present modified example, information on a plurality of consecutive pulse intervals calculated during blood pressure measurement performed in the past is stored in the storage unit 130. Hereinafter, a series of information on a plurality of pulse intervals is also referred to as “a group of pulse interval information”.

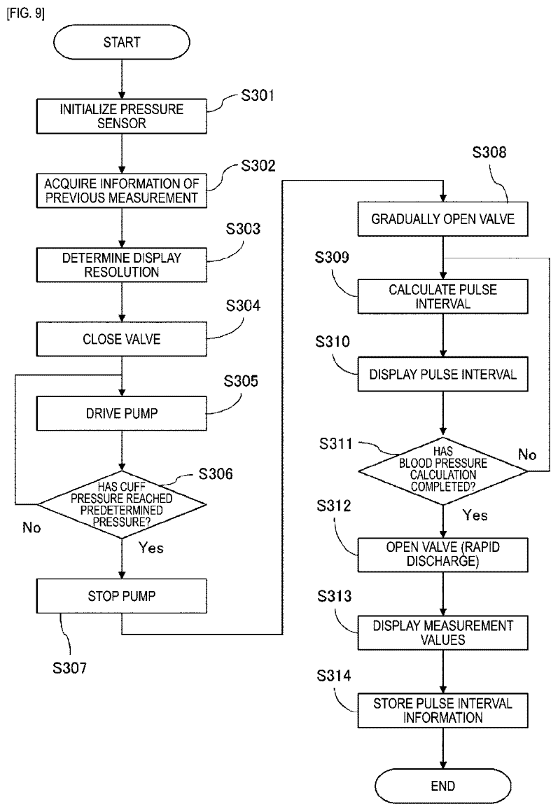

FIG. 9 illustrates a flow of processing performed when blood pressure measurement is performed by the blood pressure measurement device 1 according to the present modified example. As illustrated in FIG. 9, when the user performs an operation to start blood pressure measurement, the sensor unit 110 initializes the pressure sensor (S301). Subsequently, the display resolution determination unit 103 reads and acquires a group of pulse interval information acquired during the previous (i.e., most recent) blood pressure measurement and stored in the storage unit 130 (S302), and determines a display resolution based on this information (S303). The display resolution can be determined by the same method as that in Example 1 and Modified Example 1 or 2, and thus the description thereof is omitted.

Next, the cuff pressure control unit 120 closes the valve (S304) and drives the pump (S305) to feed air into the cuff so as to inflate the cuff. Then, the cuff pressure control unit 120 determines whether the cuff pressure has reached a predetermined pressure (e.g., 200 mHg) (S306). When it is determined that the cuff pressure has not reached the predetermined pressure, the processing returns to step S305, and the subsequent processing is repeated. On the other hand, when it is determined in step S306 that the cuff pressure has reached the predetermined pressure, the cuff pressure control unit 120 stops the pump (S307).

Following the process of step S307, the cuff pressure control unit 120 gradually opens the valve to deflate the cuff (S308). Then, the pulse interval calculation unit 102 calculates a pulse interval for each beat from a pulse wave acquired during this period (S309), and based thereon, the level indicator display content determination unit 104 determines the number of the display segments S to be activated for each beat. Then, the image display unit 150 displays the determined content at the level indicator LI1 (S310), so that the user can easily visually recognize a fluctuation in the pulse interval for each beat during the blood pressure measurement.

In addition, the blood pressure value calculation unit 101 measures blood pressure values in the course of deflation of the cuff, and in step S311, it is determined whether the blood pressure value calculation has been completed (S311). Here, when it is determined that the blood pressure value calculation has not been completed, the processing returns to step S309, and the subsequent processing is repeated. On the other hand, when it is determined in step S311 that the blood pressure value calculation has been completed, the cuff pressure control unit 120 opens the valve to rapidly discharge air from the cuff to deflate the cuff (S312). Then, the image display unit 150 displays the measurement values (S313), and subsequently, the control unit 100 executes a process of storing the group of pulse interval information calculated this time in the storage unit 130 (S314), and a series of processing ends.

As described above, in the present modified example, since the pulse interval information used for determining the display resolution is a group of pulse interval information stored during the previous blood pressure measurement, the display resolution can be set with high accuracy based on sufficient data.

### Modified Example 6

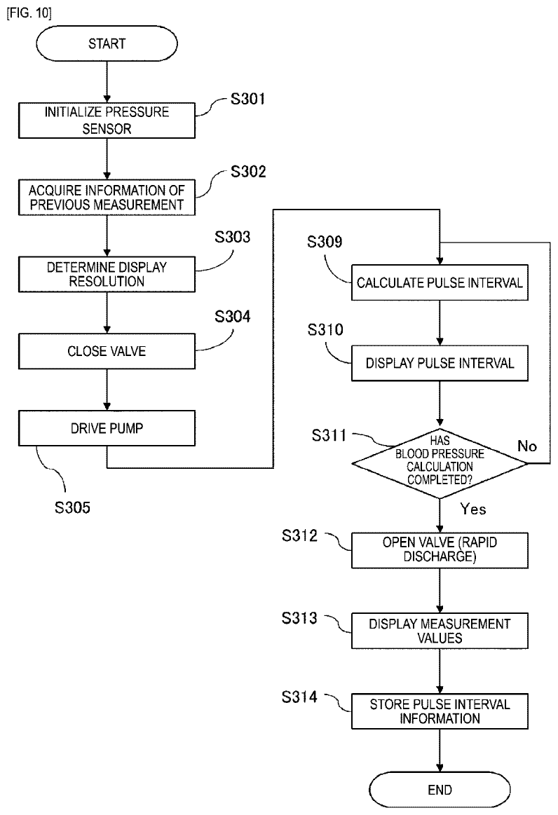

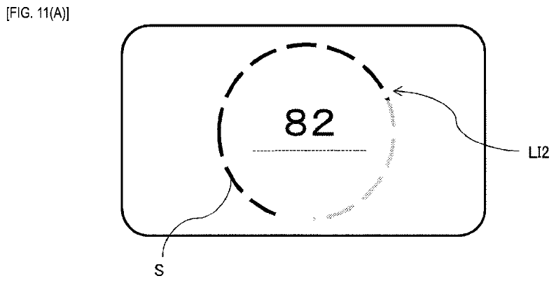

In the modified example described above, the processing performed when blood pressure values are measured by a so-called depressurization measurement method has been described, but a display resolution can be determined using a group of pulse interval information also in a process of measuring a blood pressure by a pressurization measurement method. The processing in such a case is illustrated in FIG. 10. As illustrated in FIG. 10, in the present modified example, blood pressure values are measured in the course of increasing the cuff pressure instead of using the depressurization method in which the cuff is inflated until the cuff pressure reaches a predetermined pressure and then the cuff is gradually depressurized to measure the blood pressure, and thus the processing from step S306 to step S308 are not present. Except for the above, the same processing as that in Modified Example 5 is performed. As described above, the method of determining a display resolution using a group of pulse interval information acquired in advance can be suitably used when blood pressure measurement is performed by the pressurization measurement.

### Other Points

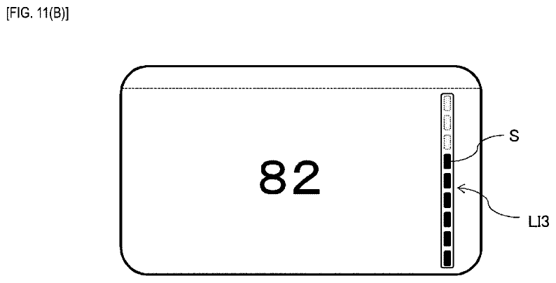

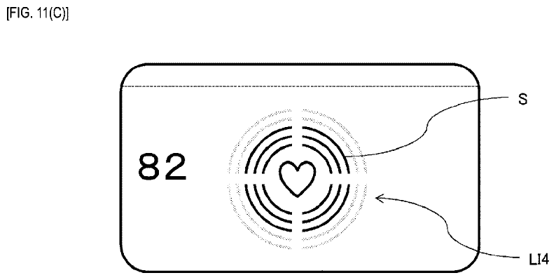

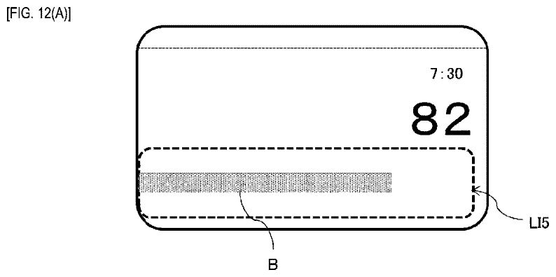

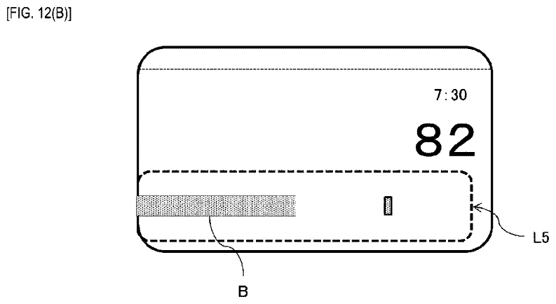

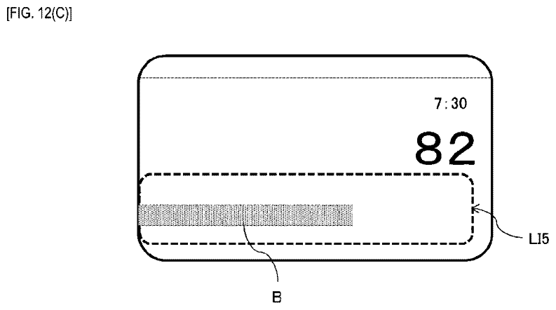

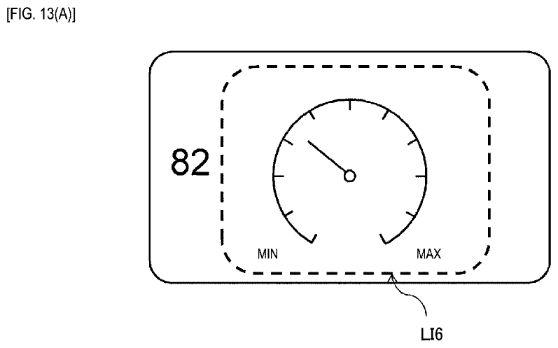

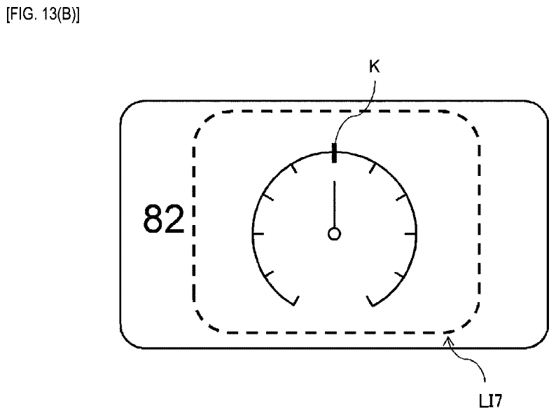

The description of the examples above is merely illustrative of the present invention, and the present invention is not limited to the specific examples described above. Within the scope of the technical idea of the present invention, various modifications and combinations may be made. For example, in the above-described examples, the level indicator LI1 is configured such that the display segments S are arranged in a straight line extending in the left-right direction, but the level indicator can be configured in various ways in accordance with the shape of the device and the structure of the image display means 151. FIG. 11(A) to FIG. 11(C), FIG. 12(A) to FIG. 12(C), and FIG. 13(A) and FIG. 13(B) illustrate display modes of level indicators according to modified examples.

The shape of the level indicator can be a circular shape, instead of a linear shape, for example, like a level indicator LI2 illustrated in FIG. 11(A). Like a level indicator LI3 illustrated in FIG. 11(B), the level indicator may be configured in a linear shape extending in the vertical direction. Further, the level indicator can be a level indicator LI4 having a mode in which the display segments S are radially arranged in a plurality of rows, and the display is activated from the inside toward the outside, as illustrated in FIG. 11(C).

The level indicator does not necessarily need to be configured with a plurality of display segments. FIG. 12(A) to FIG. 12(C) illustrate examples of a level indicator LI5 in such a case. FIG. 12(A) illustrates a state in which a continuous bar B extends from the left to the right as an indication of the pulse interval Tₓ at a certain point of time. In the case of this modified example, Nₓ may be calculated not as the number of the display segments but as the length (or area) of a display-activated region in an entire displayable region of the level indicator LI5. FIG. 12(B) illustrates a state in which the bar is sequentially hidden from the right. At this time, as in Example 1, the display of a peak level portion of the bar B in FIG. 12(A) remains activated. FIG. 12(C) illustrates a state in which the bar indicating a pulse interval extends from the left to the right and a pulse interval Tₓ+1 is displayed.

The method of representing a fluctuation in a pulse interval is not limited to an increase or decrease of the display region (the number of the display segments in which the display is activated, the length or the area of the bar) in the level indicator as described above. FIG. 13(A) and FIG. 13(B) illustrate display modes of such level indicators. The level indicators illustrated in FIG. 13(A) and FIG. 13(B) have a shape including a part of a circumference (arc) and a pointer extending from the inside of the arc toward the arc, and have a configuration like a so-called analog meter.

FIG. 13(A) is a diagram illustrating a modified example of Example 1 for indicating a pulse interval for each beat. A pointer of a level indicator LI6 is displayed so as to indicate any position between min and max on a circumference corresponding to the pulse interval for each beat. That is, the angle of the pointer changes for each beat in accordance with the pulse interval.

On the other hand, FIG. 13(B) is an example of a level indicator LI7 which is displayed so as to indicate, with reference to an average value calculated using a group of pulse interval information, a position on the arc corresponding to a deviation from the average value for each beat. Specifically, the level indicator LI7 is displayed such that a reference line K, which is disposed at a central part of the arc and indicates an average value calculated using a group of pulse interval information, is set as a standard position, and when the latest calculated pulse interval takes a value of a positive deviation from the average value, the pointer moves to the right from the reference line K by the amount of the deviation. On the other hand, when the calculated latest pulse interval takes a value of a negative deviation from the average value, the display is such that the pointer moves to the left from the reference line K by the amount of the deviation. When the calculated latest pulse interval is equal to the average value (within the same range as the average value, depending on the resolution), the pointer does not move to either the left or the right from the reference line K, but in order to make it easy to recognize that the calculated latest pulse interval is equal to the average value, a dedicated display such as blinking of the pointer may be performed.

In each of the above-described examples, an example in which the level indicator is displayed by the LCD has been described, but instead thereof, the display segments may be configured with a plurality of LED indicator lights. In such a case, turning on the LED indicator lights corresponds to the activation of the display segments.

Although a pressure pulse wave is acquired by the pressure sensor in each of the above-described examples, a volume pulse wave may be acquired by a PPG sensor. Although the blood pressure measurement device has been described as an example in each of the above examples, the present invention is not limited thereto and may be applied to other biological information measurement devices (for example, an electrocardiograph, a body composition meter, and the like) as long as the devices include a sensor capable of acquiring a pulse.

### REFERENCE NUMERALS LIST

1 Blood pressure measurement device
11 Main body portion
12 Cuff portion
13 Air tube
151 Image display means
100 Control unit
110 Sensor unit
120 Cuff pressure control unit
130 Storage unit
140 Operation unit
150 Image display unit
LI1, LI2, LI3, LI4, LI5, LI6, LI7 Level indicator
S Display segment
B Bar
K Reference line

1. A biological information measurement device comprising:
pulse acquisition unit configured to detect a pulse of a user;
pulse interval calculation units configured to calculate a pulse interval between one beat and a beat immediately before the one beat based on the pulse;
display unit configured to display a level indicator visually indicating the pulse interval; and
a display resolution determination unit configured to determine a minimum value and a maximum value of the pulse interval indicated by the level indicator using information on the pulse interval.

2. The biological information measurement device according to claim 1, wherein the display resolution determination unit determines a value obtained by multiplying a maximum value of a plurality of the pulse intervals calculated and being consecutive by a predetermined first ratio as the maximum value of the pulse interval indicated by the level indicator.

3. The biological information measurement device according to claim 2, wherein the display resolution determination unit determines a value obtained by multiplying a minimum value of a plurality of the pulse intervals calculated and being consecutive by a predetermined second ratio as the minimum value of the pulse interval indicated by the level indicator.

4. The biological information measurement device according to claim 1, wherein the display resolution determination unit calculates an average value of a plurality of the pulse intervals calculated and being consecutive, determines a value obtained by multiplying a positive maximum deviation from the average value by a third ratio as the maximum value of the pulse interval indicated by the level indicator, and determines a value obtained by multiplying a negative maximum deviation from the average value by a fourth ratio as the minimum value of the pulse interval indicated by the level indicator.

5. The biological information measurement device according to claim 1, wherein
the pulse acquisition unit includes a cuff and a pressure sensor and detects the pulse by pressurizing a blood vessel of the user with the cuff and detecting a pressure pulse wave of the blood vessel with the pressure sensor, and
the display resolution determination unit determines the minimum value and the maximum value of the pulse interval indicated by the level indicator using information on a plurality of the pulse intervals calculated from a start of pressurization of the blood vessel until a first predetermined condition is satisfied.

6. The biological information measurement device according to claim 1, further comprising storage unit configured to store information on the pulse interval detected, wherein
the pulse acquisition unit includes a cuff and a pressure sensor and detects the pulse by pressurizing a blood vessel of the user with the cuff and detecting a pressure pulse wave of the blood vessel with the pressure sensor, and
the display resolution determination unit determines the minimum value and the maximum value of the pulse interval indicated by the level indicator using the information on the pulse interval stored in the storage unit and satisfying a second predetermined condition.

7. The biological information measurement device according to claim 1, wherein
the pulse acquisition unit includes a cuff and a pressure sensor and detects the pulse by pressurizing a blood vessel of the user with the cuff and detecting a pressure pulse wave of the blood vessel with the pressure sensor, and
the display resolution determination unit determines the minimum value and the maximum value of the pulse interval indicated by the level indicator using information on a plurality of the pulse intervals being consecutive and calculated until a fluid supplied to the cuff is discharged.

8. The biological information measurement device according to claim 1, wherein the level indicator visually indicates the pulse interval by using at least any of a length, an area, an angle of a region in which display is activated on the display unit, or the number of regions in which display is activated on the display unit.

9. The biological information measurement device according to claim 8, wherein the level indicator is made up of a plurality of display segments, and represents the pulse interval by the number of the display segments in which display is activated.
